(19) United States
(12) Patent Application Publication
(10) Pub. No.: US 2007/0140978 A1
(43) Pub. Date: Jun. 21, 2007

(54) Adrenergic complement inhaler

(75) Inventors: Patrick F. Dillon, East Lansing, MI (US); Robert S. Root-Bernstein, East Lansing, MI (US)

Correspondence Address: HARNESS, DICKEY & PIERCE, P.L.C., P.O. BOX 828, BLOOMFIELD HILLS, MI, 48303, US

(73) Assignee: Board of Trustees of Michigan State University, East Lansing, MI (US)

(21) Appl. No.: 11/636,720

(22) Filed: Dec. 8, 2006

Related U.S. Application Data
(63) Continuation of application No. PCT/US05/20269, filed on Jun. 9, 2005.
(60) Provisional application No. 60/578,619, filed on Jun. 10, 2004.

(51) Int. Cl. A61K 31/375; A61K 9/12; A61K 31/355; A61K 31/198

(52) U.S. Cl. 424/45; 514/474; 514/458; 514/566

(57) ABSTRACT

Pharmaceutical compositions consisting essentially of: (a) an adrenergic complement; and (b) a pharmaceutically-acceptable carrier suitable for respiratory administration. Adrenergic complements include ascorbates, tocopherols, and polycarboxylic acid chelators. Methods are also provided for the treatment of respiratory disorders, particularly asthma.

### CROSS-REFERENCE TO RELATED APPLICATIONS

This application claims priority to International Application No. PCT/US2005/020269, filed on Jun. 9, 2005, which claims the benefit of U.S. Provisional Application No. 60/578,619, filed on Jun. 10, 2004, both of which are incorporated herein by reference.

### INTRODUCTION

This invention relates to pharmaceutical compositions and methods of enhancing respiratory function. Methods include the treatment of asthma by pulmonary administration of a composition comprising an ascorbate.

Diseases involving inflammation are characterized by the influx of certain cell types and mediators, the presence of which can lead to tissue damage and sometimes death. Diseases involving inflammation are particularly harmful when they afflict the respiratory system, resulting in obstructed breathing and lung tissue damage. Obstructive diseases of the airways are characterized by airflow limitation (i.e., airflow obstruction or narrowing) which leads to increased work in breathing, dyspnea, hypoxemia and hypercapnia. Obstructive respiratory diseases have several causes including accumulation of fluid (edema) and the hypersecretion of mucous. An additional cause of obstructive respiratory disease is the constriction of airway smooth muscle (ASM) such as the bronchial and tracheal smooth muscles.

Asthma is a chronic obstructive disease caused by airway smooth muscle inflammation. A variety of inflammatory agents can provoke airway smooth muscle constriction including allergens, cold air, exercise, infections, and air pollution. In particular, allergens and other agents in allergic or sensitized mammals (i.e., antigens and haptens) cause the release of inflammatory mediators that recruit cells involved in inflammation. (See U.S. Pat. No. 6,248,723, Irvin, issued Jun. 19, 2001.) The cellular elements that play a role in asthma include mast cells, seosinophils, T lymphocytes, macrophages, neutrophils, and epithelial cells. In susceptible individuals, airway inflammation is associated with the recurrent “asthma attacks” or “episodes” or “exacerbations” that impact normal breathing including wheezing, breathlessness, chest tightness, and coughing, particularly at night or in the early morning. (National Institutes of Health-National Heart, Lung, and Blood Institute, Expert Panel Report 2: Guidelines for the Diagnosis and Management of Asthma, NIH Publication No. 97-4051, July 1997). After time, the asthmatic becomes hyperresponsive to inflammatory agents which increases the severity of the episodes.

Asthma affects between 12 to 15 million Americans and asthma prevalence (i.e. both incidence and duration) is increasing. Of more concern, however, is the rise in the death rate. When coupled with increases in emergency room visits and hospitalizations, recent data suggests that asthma severity is rising.

Asthma can be classified into four levels or steps of severity. The levels or steps are generally based on two factors. First, the asthma is characterized based on the frequency of symptoms before treatment. Secondly, the classification is based on the decrease in breathing capacity or force of exhalation. This measurement is based on a subject's optimal and highest level of force expelled while exhaling or a “personal best.” From the personal best number, the subject's condition at any period can be measured as a “peak expiratory flow” or PEF which is a percentage of the personal best force rate. Generally, if a patient's exacerbations can be categorized into more than one level, the patient is assigned to the most severe step in which any feature occurs. Special considerations are given to young children because of the child's inability to properly conduct measuring tests.

Level 1 is mild intermittent asthma. Patients with mild intermittent asthma express symptoms less than two times each week and have nighttime symptoms less than two nights per month. The exacerbations are brief, lasting only a few hours or a few days, but between exacerbations, the patient is asymptomatic. The peak expiratory flow for mild intermittent asthmatics is generally greater than or equal to 80% of the patient's personal best. Daily medication is not required.

Level 2 is mild persistent asthma. The patients express symptoms more than twice each week (three to six days) and express nighttime symptoms more than two nights (three to four nights) per month. The exacerbation affects everyday activities. Similar to Level 1 asthmatics, the peak expiratory flow for mild persistent asthmatics is generally greater than or equal to 80% of the patient's personal best. Level 2-mild persistent asthmatics use a low dosage anti-inflammatory drug.

Level 3 is moderate persistent asthma. These patients express day time symptoms every day of the month and night time symptoms more than five nights per month. Flow rate ranges from 60% to 80% of the patient's personal best. Level 3-moderate persistent asthmatics daily medication includes any combination of medium to high dose anti-inflammatory and a long-acting bronchodilator.

Level 4 is severe persistent asthma. Level 4 asthmatics express daytime symptoms continually and the patient frequently has night time symptoms. The patient's peak expiratory flow rate is generally less than or equal to 60% of the patient's personal best. Level 4-severe persistent asthmatic's daily medication includes a high dose anti-inflammatory, a long-acting bronchodilator, and steroid tablets or syrup.

It is a goal of asthma therapy to improve respiratory function so that subjects are re-classified at a lower level. However, respiratory function can worsen, resulting in a more severe asthma level, if subjects do not comply with their therapeutic regimen (e.g., by under- or over-medication.)

In other animals, respiratory disorders manifest under similar stimuli or inflammatory agents, but are labeled with different names. For example, horses are prone to Chronic Obstructive Pulmonary Disease (COPD) or Horse Heaves. Horse Heaves are caused by an allergic response to the particles in hay dust (such as bacteria and fungi as well as tiny particles of feed grains, plants, feces, dander, and pollen). Air passage into the lungs is hindered by the contraction of smooth muscle surrounding the bronchi, bronchioles, and alveoli. The level of inflammation of the airways, coughing and mucous secretion become greater each time the horse is exposed to the hay dust. Treatment for Horse Heaves is generally preventative and utilizes environmental management to prevent or eliminate exposure to allergens. Other methods of treatment and control include use of corticosteroids and bronchodilator drugs. (Baker, Rachel, Chronic Obstructive Pulmonary Disease, Michigan State University Equine Pulmonary Laboratory, available at http://www.cvm.msu.edu/RESEARCH/PULMON/COPD.htm)

Airway smooth muscle inflammation can be controlled by catecholamines and related adrenergic (sympathomimetic) compounds. Some of these compounds are endogenous or naturally present within the subject, but can also be provided using exogenous compounds such as drugs. Catecholamines and related adrenergic compounds are diverse and useful in the treatment of a variety of clinical disorders because they directly or indirectly affect the alpha- and beta-adrenergic receptors found in tissues throughout the body.

Most of the actions of adrenergic compounds can be classified into seven broad types: (1) peripheral excitatory action on certain types of smooth muscle, such as those in blood vessels supplying skin and mucous membranes, and on gland cells, such as those in salivary and sweat glands; (2) peripheral inhibitory action on certain other types of smooth muscle, such as those in the wall of the gut, in the bronchial tree, and in blood vessels supplying skeletal muscle; (3) cardiac excitatory action, responsible for an increase in heart rate and force of contraction; (4) metabolic action such as an increase in rate of glycogenolysis in liver and muscle, and liberation of free fatty acids from adipose tissue; (5) endocrine action, such as modulation of the secretion of insulin, renin, and pituitary hormones; (6) CNS action, such as respiratory stimulation and, with some adrenergics, an increase in wakefulness, psychomotor activity, and a reduction in appetite; and (7) presynaptic actions, which result in either inhibition or facilitation of the release of neurotransmitters such as norepinephrine and acetylcholine. See, Goodman and Gilman's, The Pharmacological Basis of Therapeutics, 8^{th} Edition (1990). Disorders that can be treated using adrenergic compounds include, for example, hypertension, shock, cardiac arrhythmia, asthma, allergy, cardiac failure and anaphylaxis. The peripheral inhibitory characteristics make catecholamines and adrenergic compounds suitable for treating respiratory disorders such as asthma by relaxing airway smooth muscles.

Various factors and specific patient information can make the use of catecholamines and adrenergic compounds undesirable or a temporary remedy. The clinical use of these compounds can be undesirable and complicated, since administration may affect several different body functions. The response of a body tissue to an adrenergic compound is dictated not only by the direct effects of the compound but also by the homeostatic responses of the organism. Side effects are not uncommon, and require a careful selection of the specific adrenergic compound to be used and the dosage level in which it is to be administered.

The temporary or transitional aspects of treatment are used to prevent over medicating the patient and to decrease reliance on exogenous catecholamines and adrenergic compounds in the form of asthma related drugs. In some patients, it is a goal of therapy to “step down” the level of medication after time, and increase patient control over the disorder. For example, with a mild persistent asthmatic, the daily use of inhaled steroids gradually improves the condition and lung function and decreases the use of certain medication and the occurrence of severe episodes or exacerbations. In these patients, the use of inhaled medicine can be reduced by about 25% every two to three months. When control over the mild persistent condition is sufficient for re-classifying the patient as a mild intermittent asthmatic, the need for daily medication can be eliminated.

It would be desirable to provide a pharmaceutical composition and methods of treatment to allow the improvement or prevention of respiratory disorders by relaxing the airway smooth muscles responsible for airway constriction. It would also be desirable to allow a subject to maintain normal physical activities with minimal dosage, interference, or potential side-effects associated with using medicines for respiratory disorders. It is also desirable if the relaxation was controlled by the natural or endogenous adrenergic compounds.

### SUMMARY

The present invention provides pharmaceutical compositions for enhancing respiratory function in human or other animal subjects, including the inhalation treatment of asthma. Such compositions include those consisting essentially of:

(a) an adrenergic complement; and

(b) a pharmaceutically-acceptable carrier for respiratory administration.

Preferably, the complement is a compound selected from the group consisting of an ascorbate, a tocopherol, a polycarboxylic acid chelator, and mixtures thereof. A preferred complement is an ascorbate.

The present invention also provides various methods, including methods for the enhancement of respiratory function in a human or animal subject, comprising the pulmonary or nasal administration to said subject of a first composition consisting essentially of an adrenergic complement. In various embodiments, such methods are for the treatment or prevention of a respiratory disorder, such as nasal congestion, oral and nasal inflammation and swelling (such as caused by cold, flu, or allergies), chronic obstructive pulmonary disease, asthma, emphysema, bronchospasm, and bronchitis. In one embodiment, the present invention provides methods for the treatment or prevention of asthma in a human or animal subject, comprising the pulmonary administration to said subject of a first composition consisting essentially of an adrenergic complement. Another embodiment provides a method for treating nasal congestion, comprising the pulmonary administration of a first composition consisting essentially of an adrenergic complement. Other embodiments provide methods for the enhancement of respiratory function associated with the physical exercise of a human or animal subject having normal respiratory function, comprising the administration to said subject of an adrenergic complement.

It has been found that the compositions and methods of this invention are effective for enhancing respiratory function, including treating a broad range of respiratory disorders. Use of these methods and compositions afford advantages versus adrenergic compositions and methods among those known in the art, including enhanced efficacy, increase duration of action, reduction of side effects, and dosing flexibility. Further uses, benefits and embodiments of the present invention are apparent from the description set forth herein.

### DESCRIPTION

The following definitions and non-limiting guidelines must be considered in reviewing the description of this invention set forth herein. The headings (such as “Introduction” and “Summary”) and sub-headings (such as “Adrenergic Compound Complements” and “Methods”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

The present invention encompasses certain novel compositions and methods for the administration of adrenergic compounds to human or other animal subjects. Specific compounds and compositions to be used in the invention must, accordingly, be pharmaceutically-acceptable. As used herein, such a “pharmaceutically-acceptable” component is one that is suitable for use with humans and/or animals without undue adverse side effects (such as toxicity, irritation, and allergic response) commensurate with a reasonable benefit/risk ratio.

As referred to herein, all compositional percentages are by weight of the total composition, unless otherwise specified.

### Compositions

The present invention provides compositions consisting essentially of:

(a) an adrenergic complement; and

(b) a pharmaceutically-acceptable carrier for respiratory administration.

In various embodiments such compositions are used in therapies with an adrenergic compound, wherein (as discussed further below) the adrenergic compound is administered in a separate composition. However, such compositions do not contain clinically significant levels of adrenergic compounds. That is, adrenergic compounds, if present in such formulations, are not present at levels that afford clinical efficacy against a respiratory or other disorder either with, or without, an adrenergic complement. In other embodiments, such compositions are used in therapies without an adrenergic compound.

Adrenergic Compound Complement:

The compositions and methods of this invention comprise a compound which is a complement to an adrenergic compound. In one embodiment, a “complement” is a compound which, in a given composition or method, binds to the adrenergic compound used in said composition or method. Such “binding” is the formation of a complex through physicochemical interaction of the complement with the adrenergic compound, through means other than covalent bonding. Such bonding is described in the following articles, incorporated by reference herein: Root-Bernstein and Dillon, “Molecular Complementarity I: The Complementarity Theory of the Origin and Evolution of Life.” J. Theoretical Biology 188: 447-449 (1997); and Root-Bernstein, “Catecholamines Bind to Enkephalins, Morphiceptin, and Morphine,” Brain Research Bulletin 18: 509-532 (1987); and Root-Bernstein and Dillon, “Fostering Venture Research: A Case Study of the Discovery that Ascorbate Enhances Adrenergic Drug Activity,” Drug Development Research, 57:58-74 (2002). Such binding, and complements useful herein, are described in International Publication No. WO 02/26223, Root-Bernstein et al., published Apr. 4, 2002.

Binding between a complement and an adrenergic compound can be demonstrated through any physical, chemical, or immunological technique. Physicochemical methods include nuclear magnetic resonance imaging, ultraviolet or visible light spectroscopy, capillary or other forms of electrophoresis, high pressure liquid and other forms of chromatography, pH titration, and buffering. Chemical methods include procedures that can demonstrate binding such as affinity selection using gels, cellulose, glass, plastic, and/or other bound ligands. Immunological procedures that can demonstrate molecular complementarity include, double antibody diffusion (DAD), double antibody enzyme-linked immunosorption assay (DA-ELISA), in which antibody to the catecholamine (or agonist) and antibody to its potential complements are prepared and tested to determine whether the pairs of antibodies bind to one another.

Preferred complements include those selected from the group consisting of an ascorbate, a tocopherol, a polycarboxylic acid chelator, and mixtures thereof. The compound can also be a salt or ester of the complement. A “pharmaceutically-acceptable salt” is a cationic salt formed at any acidic (e.g., carboxyl) group, or an anionic salt formed at any basic (e.g., amino) group. Many such salts are known in the art, as described in International Publication No. WO 87/05297, Johnston et al., published Sep. 11, 1987 (incorporated by reference herein). Preferred cationic salts include the alkali metal salts (such as sodium and potassium), and alkaline earth metal salts (such as magnesium and calcium). Preferred anionic salts include the halides (such as chloride salts). A “pharmaceutically-acceptable ester” is an ester that does not essentially interfere with the activity of the compounds used herein, or that is readily metabolized by a human or lower animal subject to yield an active compound.

Ascorbates include ascorbic acid and pharmaceutical derivatives and metabolites thereof. Ascorbates include ascorbic acid, sodium ascorbate, calcium ascorbate, L-ascorbic acid, L-ascorbate, dehydrosoascorbic acid, dehydroascorbate, 2-methyl-ascorbic acid, 2-methyl-ascorbate, ascorbic acid 2-phosphate, ascorbic acid 2-sulfate, calcium L-ascorbate dihydrate, sodium L-ascorbate, ascorbylesters, and mixtures thereof. Preferred ascorbates include ascorbic acid, sodium ascorbate, calcium ascorbate, and dehydrosoascorbic acid. Ascorbic acid is a particularly preferred ascorbate.

Tocopherols include substances that have the biological and physiological activities of vitamin E, including alpha-, beta-, gamma-, delta-, epsilon-, zeta- and eta-tocopherols of natural d- and synthetic dl-forms; substituted tocols in which one, tow or three of the methyl groups in the 5, 7 and 8 positions of the chroman nucleus of tocol are replaced by a radical or radicals, such as alkyl, haloalkyl, hydroxyalkyl, aminoalkyl, acyl and allyl radicals, and in which the methyl group in the 2 position of the chroman nucleus of the tocol is replaced by a lower alkyl radical, such as ethyl, propyl and butyl; and tocopherol analogous substances such as nor-tocopherol in which the isoprene units in the 2 position of the croman nucleus are two in number, homo-tocopherol in which the isoprene units are four in number and iso-tocopherol in which a side chain composed of three isoprene units in the tocol is linear. Tocol has the structure of 2-methyl-2-[trimethyltridecyl]-6-hydroxychromane. (See U.S. Pat. No. 3,122,565, Kijima, et al., issued Feb. 25, 1964 and U.S. Pat. No. 4,550,183, Willging, issued Oct. 29, 1985). A preferred tocopherol is alpha-tocopherol.

Polycarboxylic acid chelators include ethylenediamine tetraacetic acid (EDTA), diethylene triamine pentaacetic acid, pharmaceutically-acceptable salts thereof, and mixtures thereof. A preferred polycarboxylic acid chelator is EDTA.

Carrier:

The compositions of the present invention comprise a pharmaceutically-acceptable carrier, which is preferably suitable for respiratory delivery. As referred to herein, “respiratory delivery” means topical administration of the adrenergic complement to the mucosa of the respiratory tract, including nasal and pulmonary administration. Carrier materials among those useful herein include diluents, buffering agents, and solvents. Formulations suitable for respiratory administration include compositions of the adrenergic complement compounds in a form that can be dispensed by inhalation or nasal spray devices among those known in the art. Embodiments include liquid or powdered compositions suitable for nebulization and intrabronchial use, or aerosol compositions administered via an aerosol unit dispensing metered doses. Suitable liquid compositions comprise the active ingredient in an aqueous, pharmaceutically-acceptable inhalant solvent, e.g., isotonic saline or bacteriostatic water. The solutions are administered by means of a pump or squeeze-actuated nebulized spray dispenser, or by any other means for causing or enabling the requisite dosage amount of the liquid composition to be sprayed into the nose or inhaled into the lungs. Devices used to deliver the pharmaceutical composition include nebulizers, aspirators, inhalers, and nasal sprays.

The compositions optionally comprise a non-adrenergic active material, preferably having efficacy for the treatment or prevention of a respiratory disorder, such as nasal congestion, oral and nasal inflammation and swelling (such as caused by cold, flu, or allergies), chronic obstructive pulmonary disease, asthma, emphysema, bronchospasm, and bronchitis. Non-adrenergic actives among those useful herein include steroidal and non-steroidal anti-inflammatories. Preferred anti-inflammatories include beclomethasone, budesonide, flunisolide, fluticasone, triamcinolone acetonide, and mixtures thereof.

Techniques and compositions for making dosage forms useful in the methods of this invention are described in the following references, all incorporated by reference herein: 7 Modern Pharmaceutics, Chapters 9 and 10 (Banker & Rhodes, editors, 1979); Lieberman et al., Pharmaceutical Dosage Forms: Tablets (1981); Ansel, Introduction to Pharmaceutical Dosage Forms 2d Edition (1976); and U.S. Pat. No. 5,646,139, White et al., issued Jul. 8, 1997.

The compositions of the present invention comprise an adrenergic complement at a level sufficient to deliver a safe and effective amount of complement when used in a method of this invention. The specific level will depend on a variety of factors, including the specific complement, the specific carrier formulation, and the delivery device (if any) to be used for administration. Preferably, the composition delivers the adrenergic complement to the respiratory tissues to which they are applied (e.g., nose, throat or lungs) of a human or animal subject, so as to raise the local concentration of the complement in the tissue to levels above that present in the serum of the subject. Preferably, the local concentration in the respiratory tissue is greater than that obtained by oral administration of the complement. In various embodiments, the adrenergic complement is preferably present at a level of from about 0.1% to about 90% (by weight of the composition), optionally in various embodiments at levels of from about 1% to about 80%, or from about 10% to about 50%.

In one embodiment, a composition of the present invention comprises a time-release formulation of an adrenergic complement, such as a time-release formulation of an ascorbate. In one embodiment, the composition comprises an ascorbate selected from the group consisting of ascorbate phosphate, ascorbate sulfate, and mixtures thereof.

In one embodiment, the composition comprises a hyperpreserving amount of the complement, such as a hyperpreserving amount of an ascorbate, a hyperpreserving amount of a tocopherol, a hyperpreserving amount of a polycarboxylic acid chelator or a mixture thereof. As referred to herein, a “hyperpreserving amount” of an ascorbate, a tocopherol, a polycarboxylic acid chelator, or mixtures thereof is an amount that is in excess of the amount of such material that is conventionally used (the “preservative level”) to preserve a pharmaceutical active material (e.g., an adrenergic compound) in a pharmaceutical dosage form (e.g., to prevent the oxidation of the pharmaceutical active compound in solution). Preferably, the preservative level of the complement is that amount which is demonstrated to protect an adrenergic active compound in a clinical dosage form from degradation over a reasonable shelf life (e.g., two years) under typical storage conditions. Preferably the preservative level is that which is demonstrated in the art to have preservative utility in compositions comprising adrenergic compounds, preferably at levels approved for commercial marketing of such products. In such embodiments of this invention, the dosage forms of this invention comprise a concentration of complement at least about 10, preferably at least about 25, preferably at least about 50, preferably at least about 100, preferably at least about 150, preferably about 200, times higher than the concentration amount needed to preserve an adrenergic compound.

In various embodiments, the compositions of the present invention are provided in a device that facilitates administration of the adrenergic complement in a unit dosage. As used herein, a “unit dose” of a composition of this invention comprises an amount of an adrenergic complement compound that is suitable for administration to a human or lower animal subject, in a single dose, according to good medical practice.

The specific safe and effective amount of the adrenergic complement will vary with such factors as the particular condition being treated, the physical condition of the patient, the nature of concurrent therapy (if any), the specific adrenergic complement used, the specific route of administration and dosage form, the carrier employed, and the desired dosage regimen. In one embodiment, the complement is administered at a level effective to potentiate endogenous adrenergic compounds in a human or other animal subject to which the composition is administered. In one embodiment, the complement is administered at a level effective to potentiate the activity of an adrenergic compound that is co-administered to the subject.

Devices suitable for administering unit doses include those known in the art. Such devices include nebulizers, aspirators, inhalers, and nasal sprayers. Nebulizers work by forming aerosols or converting bulk liquid into small droplets suspended in a breathable gas. In particular, the nebulizers for use herein nebulize liquid formulations of the compositions provided herein. The nebulizer may produce the nebulized mist by any method known to those of skill in the art, including, but not limited to, compressed air, ultrasonic waves, or vibration. The nebulizer may further have an internal baffle. The internal baffle, together with the housing of the nebulizer, selectively removes large droplets from the mist by impaction and allows the droplets to return to the reservoir. The fine aerosol droplets thus produced are entrained into the lung by the inhaling air/oxygen. (See, e.g., U.S. Pat. No. 6,667,344, Banerjee, et al., issued Dec. 23, 2003; U.S. Pat. No. 6,340,023, Elkins, issued Jan. 22, 2002; U.S. Pat. No. 5,586,551, Hilliard, issued Dec. 24, 1996; U.S. Pat. No. 5,355,872, Riggs, et al., issued Oct. 18, 1994; U.S. Pat. No. 5,186,166, Riggs, et al., issued Feb. 16, 1993; and U.S. Pat. No. 4,865,027, Laanen et al., issued Sep. 12, 1989.)

Exemplary inhalers include metered dose inhalers and dry powdered inhalers. A metered dose inhaler or “MDI” is a pressure resistant canister or container filled with a product such as a pharmaceutical composition dissolved in a liquefied propellant or micronized particles suspended in a liquefied propellant. The correct dosage of the pharmaceutical composition is delivered into the patient's oropharnyx. (See, e.g., U.S. Pat. No. 5,544,647, Jewett et al., issued Aug. 13, 1996.)

A dry powder inhaler is a system operable with a source of pressurized air to produce dry powder particles of a pharmaceutical composition that is compacted into a very small volume. For inhalation, the system has a plurality of chambers or blisters each containing a single dose of the pharmaceutical composition and a select element for releasing a single dose (See, e.g., U.S. Pat. No. 6,642,275, Alfonso, et al., issued Nov. 4, 2003; U.S. Pat. No. 6,626,173, Genova, et al., issued Sep. 30, 2003; U.S. Pat. No. 5,694,920, Abrams, et al., issued Dec. 9, 1997; and U.S. Pat. No. 5,033,463, Cocozza, issued, Jul. 23, 1991.)

Suitable powder compositions include, by way of illustration, powdered preparations of the active ingredients thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants and co-solvents and may be filled into conventional aerosol containers that are closed by a suitable metering valve.

Another embodiment of the invention is a nasal spray. Preferred nasal sprays are in liquid form such as an aqueous solution or suspension, an oil solution or suspension, or an emulsion, depending on the properties of the composition components. Optional ingredients ensure minimal irritation, proper spray composition, and adequate delivery. Buffers such as citrate, phosphate, and glycine adjust the pH of the nasal spray to prevent irritation to the nose. Moisturizing agents such as propylene glycol and glycerine are also useful in the nasal spray. Other optional ingredients such as polyphosphoesters, polyethylene glycol, high molecular weight polylactic acid, microsphere encapsulations such as polyvinylpyrrolidone, hydroxypropyl cellulose, chitosan, and polystyrene sulfonate enhance the retention time of the composition. The composition may be administered in any of a variety of devices, including nasal sprayers among those known in the art. In one embodiment, the nasal spray is delivered in a non-pressurized dispenser that provides a metered dose of the adrenergic complement.

### Methods of Treatment

This invention also provides methods of improving respiratory function in a human or other animal subject comprising the respiratory administration of an adrenergic complement to said subject. In one embodiment, for the treatment or prevention of a respiratory disorder, the subject is administered a first composition consisting essentially of an adrenergic complement including the adrenergic complement, as described above. In a preferred embodiment, the subject is administered a complement from the group consisting of ascorbic acid, sodium ascorbate, calcium ascorbate, dehydrosoascorbic acid, alpha tocopherol, and mixtures thereof.

The specific dosage of compounds to be administered, as well as the duration of treatment, is mutually dependent. The dosage and treatment regimen will also depend upon such factors as the specific purpose of the method (e.g., the specific disorder to be treated or prevented), the complement used, the ability of the complement to reach efficacious concentrations at the site of the action, the nature and extent of other disorders (if any), the personal attributes of the subject (such as weight), compliance with the treatment regimen, the nature of concomitant therapies (if any), and the presence and severity of any side effects of the method.

In one embodiment, the methods comprise administration of from about 0.2 to about 500 mg ascorbic acid. In embodiments for administration to a human, the dosage of ascorbic acid is preferably from about 0.5 mg to about 30 mg, optionally from about 1 mg to about 20 mg, from about 2 mg to about 15 mg, or from about 3 mg to about 10 mg, preferably in a unit dose. In one embodiment, the methods comprise administration of from about 0.3 mg to about 750 mg EDTA. In embodiments for administration to a human, the dosage is preferably from about 0.7 mg to about 45 mg, optionally from about 2 mg to about 30 mg, from about 3 mg to about 20 mg, or from about 5 mg to about 15 mg, preferably in a unit dose. In one embodiment, the methods comprise administration of from about 0.01 mg to about 200 mg, preferably from about 0.1 mg to about 50 mg, alpha-tocopherol. In embodiments for administration to a human, the dosage of alpha-tocopherol is preferably from about 0.1 mg to about 5 mg, optionally from about 0.1 mg to about 2 mg, or from about 0.1 mg to about 1 mg, preferably in a unit dose.

In various embodiments, the present invention provides methods for the treatment or prevention of a respiratory disorder. Such disorders include disorders that are mediated by adrenergic receptors, such as nasal congestion, oral and nasal inflammation and swelling (such as caused by cold, flu, or allergies), chronic obstructive pulmonary disease, asthma, emphysema, bronchospasm, and bronchitis. Preferred methods include those for treatment or prevention of asthma and nasal congestion.

As discussed above, asthma may be the result of any of a variety conditions, including allergy and other environmental triggers. Such “triggers” include agents that provoke airflow inflammation and can cause an attack. Triggers include, but are not limited to, allergens, cold air, exercise, infections, air pollution, genetic sensitivity, and microorganisms such as bacteria and fungus. In one embodiment, the present invention provides administration of an adrenergic complement following the inception of an asthma attack. Such an attack is an acute response due to the inflammation of airways, manifested by such symptoms as wheezing, breathlessness, chest tightness, and coughing at any time or specifically during the morning and/or evening. Such responses include those due to inflammation following a specific trigger or stimuli. In one embodiment the present invention provides a method for the treatment of asthma, including the increase of peak expiratory airflow after the inception of an asthma attack. In another embodiment, the present invention provides a method for the prevention of asthma, including the prevention of an asthma attack after exposure of a susceptible individual to an asthma trigger.

In various embodiments, the present invention provides methods of treating a human subject having mild intermittent or mild persistent asthma, as determined according to National Institutes of Health Guidelines (National Institutes of Health-National Heart, Lung, and Blood Institute, Expert Panel Report 2: Guidelines for the Diagnosis and Management of Asthma, NIH Publication No. 97-4051, July 1997). As used herein, a “Level 1” or “mild intermittent” asthmatic has brief exacerbations that are asymptomatic between episodes. Level 1 asthmatics experience symptoms around two times each week and have nighttime symptoms less than two nights per month. The peak expiratory flow for a mild intermittent asthmatic is generally greater than or equal to 80% of his or her personal best. Daily medication is not required. As used herein, “Level 2” or “mild persistent asthma” includes subjects that express symptoms more than twice each week (3-6 days) and express nighttime symptoms more than two nights (3-4 nights) per month. Similar to level 1 asthmatics, the peak expiratory flow for a mild persistent asthmatic is generally greater than or equal to 80% of his or her personal best. Level 2-mild persistent asthmatics conventionally use a low dosage anti-inflammatory drug. In one embodiment, the present invention provides methods comprising the administration of an adrenergic complement for sufficient time so as to improve the severity of asthma, such as for improving a Level 2 asthma to Level 1. With the classification of asthmatics, it is understood that individuals may be placed in multiple categories. It is understood that categorization is based on the patient's most intense symptoms. Although the classification standards are based on the standards from the National Institutes of Health, it is understood that patients experiencing symptoms but classified under another system can still utilize the compositions and methods of embodiments of this invention.

In various embodiments, the present invention provides a method consisting essentially of administering an adrenergic complement to a subject having asthma, in a particular one having mild asthma. In such a method, the subject is not administered an adrenergic compound. In one such method, the subject is administered the adrenergic complement prophylactically, so as to diminish the number of asthma episodes, the intensity of episodes, duration of episodes, or combinations thereof.

The present invention also provides methods for preventing or treating nasal congestion. Nasal congestion may be caused by any of a variety of factors, including allergic rhinitis, bacterial infection, and viral infection (such as cold and flu). In one embodiment, the method consists essentially of administering an adrenergic complement. In such a method, the subject is not administered an adrenergic compound.

In various embodiments, the methods comprise administering a first composition consisting essentially of an adrenergic complement by respiratory delivery, and administering a second composition comprising an adrenergic compound. In one embodiment, such methods are for the treatment of asthma, wherein the adrenergic complement is administered by inhalation. As referred to herein, “first” and “second” do not limit the order of administration of the compositions. Thus, in one embodiment, the first composition is administered first and the second composition is later administered. In another embodiment, the second composition is administered first. In another embodiment, both compositions are administered substantially concurrently.

Adrenergic compounds useful herein include pharmaceutically-acceptable compounds which directly or indirectly agonize or antagonize an alpha- or beta-receptor, eliciting a sympathomimetic response. Many adrenergic compounds are known in the art, including those described in Goodman and Gillman's, The Pharmacological Basis of Therapeutics, 8^{th} Edition (1990) (incorporated by reference herein). Adrenergic compounds useful herein include those selected from the group consisting of albuterol, amantadine, amphetamine, benzephetamine, bitolterol, clonidine, colterol, dextroamphetamine, diethylpropion, dobutamine, dopamine, ephedrine, epinephrine, ethylnorepinephrine, fenfluramine, fenoterol, guanabenz, guanfacine, hydroxyamphetamine, isoetharine, isoproterenol, levodopa, mephenxermine, metaproterenol, metaraninol, methamphetamine, methoxamine, methyldopa, methylphendate, norepinephrine, oxymetazoline, pemoline, phendimetrazine, phenmetrazine, phentermine, phenylephrine, phenylethylamine, phenylpropanolamine, pirbuterol, prenalterol, propylhexedrine, pseudoephedrine, ritodrine, terbutaline, theophylline, tyramine, and derivatives thereof, pharmaceutically-acceptable salts and esters thereof, and mixtures thereof. Preferred adrenergic compounds include catecholamines, comprising molecules with a catechol (dihydroxybenzene) moeity. Particularly preferred catecholamines include those selected from the group consisting of albuterol, ephedrine, epinephrine, norepinephrine, oxymetazoline, phenylephrine, phyenylpropanolamine, pseudoephrine, theophiline, and mixtures thereof.

In one embodiment, methods of this invention preferably comprise the administration of the adrenergic compound complement at “synergistic” levels with administration of the adrenergic compound. Such effects include one or more of increasing the effect of the adrenergic compound, increasing the duration of the effect of the adrenergic compound, and making adrenergic compounds effective at dosage levels that would otherwise be ineffective.

The second composition, comprising the adrenergic compound, may be administered orally, parenterally or topically through nasal or pulmonary delivery. The second composition optionally comprises other active materials. In one embodiment, the second composition additionally comprises an adrenergic complement, which may or may not be the same as the complement present in the first composition.

In various embodiments, the methods of this invention also comprise administering a non-adrenergic active. Non-adrenergic actives among those useful herein include steroidal and non-steroidal anti-inflammatories and analgesics. Preferred anti-inflammatories include beclomethasone, budesonide, flunisolide, fluticasone, triamcinolone acetonide, and mixtures thereof. Such non-adrenergic actives may be administered as part of the first composition, or as a second composition (which may, or may not, include an adrenergic compound). Analgesics including, but not limited to opiate derivatives, codeine and morphine, are also suitable non-adrenergic actives. In various embodiments, the analgesic and adrenergic complement combination can be particularly useful in treating bronchitis by increasing air flow, decreasing congestion, improving mucus elimination, and reducing mucus production. In one embodiment, the non-adrenergic active, such as an anti-inflammatory, is administered topically, through nasal or pulmonary administration.

The present invention also provides methods for enhancing the respiratory function of “normal” human or animal subjects. Such subjects include, in various embodiments, those that do not have asthma, nasal decongestion, or other respiratory disorder. Such subjects not having asthma have a peak expiratory flow rate of greater than 90%. In one embodiment, the method comprises administration of a composition comprising an adrenergic complement. In another embodiment, the method comprises administration of a composition consisting essentially of an adrenergic complement (as discussed above).

In one embodiment, the method comprises enhancing respiratory function of a human or animal subject by administering an adrenergic complement. Animal subjects include working animals and companion animals. Such exertion includes racing, agricultural uses, and pulling carriages or other means of conveyance. Physical exertion in a human includes non-sedentary activities such as exercising (walking, running, aerobics, and swimming) and household activities or chores such as gardening, sweeping, dusting, and polishing furniture or performing errands. Various embodiments include athletic activities such as volleyball, running, hiking, rock climbing, basketball, racquetball, football, baseball, soccer, and golf. The complement is administered at any point before, after, or during the activity. In one embodiment, the complement is administered prior to commencing physical exertion.

The following non-limiting examples illustrate the compositions and methods of the present invention.

### EXAMPLE 1

A composition of the present invention is made having the following components:

| Component | Weight |
|---|---|
| hydrofluoroalkane propellant | QS |
| ascorbic acid | 0.1 g |

The propellant is in a quantity sufficient to fill a 200 puff inhaler canister. A level 2 mild persistent asthmatic is prescribed 8 to 12 daily puffs from a beclomethasone diproprionate inhaler (42 μg/puff) to control exacerbations. After a treatment period of 9 months, the patient shows decreased exacerbations and requires only 2 to 4 daily puffs of beclomethasone dipropionate for control and relief. Due to the increased control, the patient is re-classified as a level 1 mild intermittent asthmatic. The subject then begins a regimen according to the present invention, by inhaling three puffs daily from the ascorbic acid inhaler of this Example, (500 μg/puff), and utilizing an albuterol short acting bronchodilator (90 μg/puff) only during asthmatic episodes, prior to exercise, or prior to exposure to allergens. The patient is able to control exacerbations and has a decreased reliance on adrenergic asthma drugs.

### EXAMPLE 2

To combat the anticipated breathing difficulties associated with changes in altitude, a hiker (with normal respiratory function) inhales the composition of Composition 1, taking one puff every four hours. The subject observes significantly improved ease in breathing.

### EXAMPLE 3

A composition of the present invention is made having the following components:

| Component | Weight Percentage |
|---|---|
| isotonic saline | 89% |
| calcium ascorbate | 7.2% |
| EDTA | 3.8% |

On an annual basis, a horse shows an increased cough during the spring season due to pollen. A nebulizer is used to administer the composition of this Example. This treatment is administered for a period of 25 minutes each day of the spring season. The seasonal induced cough is controlled.

### EXAMPLE 4

A composition of the present invention is made having the following components:

| Component | Weight Percentage |
|---|---|
| water | 89.6025% |
| sodium phosphate dibasic | 0.0975% |
| sodium phosphate monobasic | 0.55% |
| polyvinylpyrrolidone | 1.25% |
| polyethylene glycol | 3.5% |
| dehydrosoascorbic acid | 5.0% |

A ten-year old child presents with severe nasal congestion due to a cold. The child is treated with an oral over-the-counter medication containing pseudophedrine, administered every four hours, obtaining limited relief. The child then supplements the oral medication by using the composition of this Example, one spray into each nostril, following each dose of the oral medication. Significant improvement is seen in congestion.

### EXAMPLE 5

A human subject suffering from chronic obstructive pulmonary disease is administered an oral composition comprising ephedrine and a composition of this invention comprising a metered aerosol of 0.9% saline which delivers ascorbic acid at a unit dosage of 5 mg per dose. The subject observes significant improvement in respiratory function.

What is claimed is:

1. A pharmaceutical composition for the respiratory function, consisting essentially of:
(a) an adrenergic complement; and
(b) a pharmaceutically-acceptable carrier suitable for respiratory administration.

2. A composition according to claim 1, wherein said adrenergic complement is selected from the group consisting of an ascorbates, tocopherols, a polycarboxylic acid chelator, and mixtures thereof.

3. A composition according to claim 2, wherein said adrenergic complement is selected from the group consisting of ascorbates, tocopherols, and mixtures thereof.

4. A composition according to claim 3, wherein said adrenergic complement is selected from the group consisting of ascorbic acid, sodium ascorbate, calcium ascorbate, dehydrosoascorbic acid, alpha tocopherol, and mixtures thereof.

5. A composition according to claim 4, wherein said adrenergic complement is ascorbic acid.

6. A composition according to claim 2, wherein said adrenergic complement is EDTA.

7. A composition according to claim 2, comprising a complement at a level effective to potentiate the endogenous adrenergic compounds in a human or other animal subject to whom the composition is administered.

8. A composition according to claim 7, comprising a complement selected from the group consisting of a hyperpreserving amount of an ascorbate, a hyperpreserving amount of a polycarboxylic acid chelator, and mixtures thereof.

9. A composition according to claim 8, comprising a hyperpreserving amount of an ascorbate.

10. A composition according to claim 1, wherein said carrier is operable for administration with a device selected from the group consisting of nebulizers, aspirators, inhalers, and nasal sprayers.

11. A composition according to claim 10, wherein said carrier is operable for administration with an inhaler.

12. A composition according to claim 11, further comprising a propellant.

13. A composition according to claim 10, wherein said carrier is operable for administration with a nasal sprayer.

14. A method for the treatment or prevention of asthma in a human or animal subject, comprising the pulmonary administration to said subject of a first composition consisting essentially of an adrenergic complement.

15. A method according to claim 14, wherein said adrenergic complement is selected from the group consisting of an ascorbate, a tocopherol, a polycarboxylic acid chelator, and mixtures thereof.

16. A method according to claim 15, wherein said adrenergic complement is selected from the group consisting of ascorbates, tocopherols, and mixtures thereof.

17. A method according to claim 16, wherein said complement comprises ascorbic acid administered single unit dose at a level of about 1 mg to about 10 mg.

18. A method according to claim 14, wherein said subject has intermittent or persistent mild asthma.

19. A method according to claim 14, wherein said first composition is administered using an inhaler.

20. A method according to claim 14, for the prevention of an asthma attack.

21. A method according to claim 14, additionally comprising the administration to said subject of a second composition comprising a compound selected from the group consisting of anti-inflammatory compounds, adrenergic compounds, and mixtures thereof.

22. A method according to claim 21, wherein said second composition comprises an adrenergic compound selected from the group consisting of albuterol, ephedrine, epinephrine, norepinephrine, oxymetazoline, phenylephrine, phenylpropanolamine, pseudoephedrine, theophylline, and mixtures thereof.

23. A method according to claim 21, comprising pulmonary administration of said second composition.

24. A method according to claim 21, wherein said second composition additionally comprises an adrenergic complement.

25. A method according to claim 24, wherein said adrenergic complement is the same as the adrenergic complement of said first composition.

26. A method for the enhancement of respiratory function of a human or animal subject, comprising the pulmonary administration to said subject of a first composition consisting essentially of an adrenergic complement.

27. A method according to claim 26, comprising pulmonary administration of said first composition.

28. A method according to claim 26, for the treatment or prevention of a pulmonary disorder.

29. A method according to claim 28, wherein said subject has intermittent or persistent mild asthma.

30. A method according to claim 28, additionally comprising the administration to said subject of a second composition comprising a compound selected from the group consisting of anti-inflammatory compounds, adrenergic compounds, and mixtures thereof.

31. A method according to claim 26, wherein said subject has a peak expiratory flow rate of greater than 90%.

32. A method according to claim 26, wherein said administration is associated with physical exertion by said subject.

33. A method according to claim 32, wherein said subject has a peak expiratory flow rate of greater than 90%.

34. A method according to claim 32, wherein said physical exertion is an athletic activity.

35. A method according to claim 33, comprising administration of said complement at a level effective to improve endurance of said subject while performing said physical exertion.

36. A method according to claim 31, comprising administering said first composition prior to commencing said physical exertion.

37. A method according to claim 32, wherein said subject is a human.

38. A method according to claim 32, wherein said subject is an animal.

39. A method for the enhancement of respiratory function associated with the physical exercise of a human or animal subject having normal respiratory function, comprising the administration to said subject of a compound selected from the group consisting of an ascorbate, a tocopherol, a polycarboxylic acid chelator, and mixtures thereof.

40. A method according to claim 39, consisting essentially of said administration of said compound.

41. A method according to claim 39, wherein said administration is oral.

42. A method according to claim 39, wherein said administration is pulmonary.

43. A method for the treatment or prevention of nasal congestion in a human or animal subject, comprising the nasal administration to said subject of a first composition consisting essentially of an adrenergic complement.

44. A method according to claim 43, wherein said adrenergic complement is selected from the group consisting of an ascorbate, a tocopherol, a polycarboxylic acid chelator, and mixtures thereof.

45. A method according to claim 44, wherein said adrenergic complement is selected from the group consisting of ascorbates, tocopherols, and mixtures thereof.
